(19) United States
(12) Patent Application Publication
(10) Pub. No.: US 2026/0323844 A1
(43) Pub. Date: Oct. 15, 2026

(54) STRETCHABLE ELECTRONICS FROM MULTI-LAYER FLEX LASER POST-PROCESSING

(71) Applicant: Cornell University, Ithaca, NY (US)

(72) Inventors: Simon Dunham, New York, NY (US); Bobak Mosadegh, New York, NY (US)

(73) Assignee: Cornell University, Ithaca, NY (US)

(21) Appl. No.: 19/476,110

(22) PCT Filed: Apr. 19, 2024
(86) PCT No.: PCT/US2024/025480

§ 371 Date: Oct. 16, 2025

Related U.S. Application Data
(60) Provisional application No. 63/460,856, filed on Apr. 20, 2023.

(51) Int. Cl. H05K 3/46; H05K 1/02; H05K 3/28

(52) U.S. Cl. CPC H05K 3/4644; H05K 1/028; H05K 3/28

(57) ABSTRACT

The disclosure includes example processes for creating stretchable electronics from multi-layer flex printed circuit boards (flex-PCBs), and example platforms for soft robotic sensor arrays (SRSAs) for electric mapping applications, such as cardiac voltage mapping.

### CROSS-REFERENCE TO RELATED APPLICATIONS

This application claims the benefit of and priority to U.S. Provisional Application No. 63/460,856 filed Apr. 20, 2023, the contents of which are herein incorporated by reference in their entirety for any and all purposes.

### STATEMENT REGARDING FEDERAL FUNDING

This invention was made with government support under 5 R61 HL156153-02 awarded by the National Institutes of Health. The government has certain rights in the invention.

### TECHNICAL FIELD

Embodiments of the present technology correspond to systems and methods related to stretchable electronics, such as soft robotic sensor arrays (SRSAs) and SRSA actuators, and fabrication of devices that can incorporate multiple sensors for signal acquisition, for such applications as mapping of bioelectrical activity.

### BACKGROUND

Stretchable electronics allow for more conformable mapping of electrical signal activity. Enhancements to such electronics can lead to a more accurate and detailed map of conditions such as cardiac arrhythmia.

### SUMMARY

In various embodiments, this disclosure relates generally to, for example, creating stretchable electronics, to optimizing or otherwise enhancing the post-processing of commercial multi-layer flex PCB to yield stretchable electronics, and to stretchable electronics from dual-layer flex-PCB that can include serpentine sensor array designs. The disclosure can apply to mapping of electrical activity, such as cardiac or neurological electrical activity.

These stretchable electronics can be used as a platform for soft robotic sensor arrays (SRSA) for bioelectrical mapping. There is a crucial need for devices that can embed multiple sensors and provide better signal acquisition such applications as cardiac mapping. Various embodiments provide for a fabrication process of a dual-layer multielectrode flex-PCB soft robotic sensor array (SRSA). The fabrication can use self-aligned laser-postprocessed in multi-layer flex-PCB materials. Arrays disclosed herein can be utilized for cardiac mapping by, for example, showing the SRSA's ability to acquire electrocardiograms both in vitro as well as in vivo on a leporine heart.

These and other aspects and implementations are discussed in the non-limiting examples discussed below and in an Appendix or one or more Appendices (which include drawings). The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form. For example, by appropriate computer programs, which can be carried on appropriate carrier media (computer readable media), which can be tangible carrier media (e.g. disks) or intangible carrier media (e.g. communications signals). Aspects can also be implemented using suitable apparatus, which can take the form of programmable computers running computer programs arranged to implement the aspect. As used in the specification and in the claims, the singular form of ‘a’, ‘an’, and ‘the’ include plural referents unless the context clearly dictates otherwise.

In one aspect, various embodiments relate to a method of manufacturing a device. The method may comprise: sandwiching a sacrificial layer between a first non-sacrificial layer and a second non-sacrificial layer; encapsulating, in a third non-sacrificial layer, a laser postprocessed flexible printed circuit board (flex-PCB) comprising one or more electrodes, wherein the third non-sacrificial layer comprises one or more windows to expose the one or more electrodes; bonding the first non-sacrificial layer, the second non-sacrificial layer, and the third non-sacrificial layer together; and sacrificing the sacrificial layer.

In various embodiments, the device is a soft robotic sensor array (SRSA) actuator. In various embodiments, the flex-PCB comprises a conductive trace between two insulating layers. In various embodiments, the method comprises postprocessing the flex-PCB. In various embodiments, postprocessing the flex-PCB comprises applying a laser to remove a first portion of the insulating layer without removing a second portion of the insulating layer. In various embodiments, postprocessing the flex-PCB comprises applying a first laser power level at a first region of the flex-PCB and applying a second laser power level at a second region of the flex-PCB. In various embodiments, postprocessing the flex-PCB comprises subjecting the flex-PCB to ultrasonication. In various embodiments, the sacrificial layer is a water-soluble layer. In various embodiments, the method comprises infusing the bonded first, second, and third non-sacrificial layers in water to dissolve the sacrificial layer. In various embodiments, encapsulating the flex-PCB in the third non-sacrificial layer comprises permanent adhesion by chemical means, heat-pressing, and/or welding the third non-sacrificial layer to the flex-PCBPCB. In various embodiments, the method comprises laser-cutting the one or more windows in the third non-sacrificial layer. In various embodiments, bonding the first, second, and third non-sacrificial layers comprises permanent adhesion by chemical means, heat-pressing, and/or welding the first, second, and third non-sacrificial layers together.

In another aspect, various embodiments relate to a method of postprocessing a multilayer flexible PCB (flex-PCB). The postprocessing may be performed to enhance deformability of the flex-PCB. Deformability may be increased the allow the flex-PCB to accommodate actuation. The flex-PCB may comprise a conductive trace at least partially embedded in an insulating layer. The method may comprise applying a laser to the flex-PCB so as to remove a first portion of the insulating layer without removing a second portion of the insulating layer, wherein the second portion of the insulating layer is below the conductive trace.

In various embodiments, applying the laser to the flex-PCB comprises applying a first laser power at a first region of the flex-PCB and a second laser power at a second region of the flex-PCB. In various embodiments, the method comprises subjecting the flex-PCB to ultrasonication prior to applying the laser to the flex-PCB.

In yet another aspect, various embodiments relate to a device comprising: a plurality of elastomer layers, the plurality of elastomer layers comprising a first elastomer layer with one or more laser-cut windows; and a laser postprocessed flexible printed circuit board (flex-PCB) encapsulated in the plurality of elastomer layers, wherein the flex-PCB includes one or more electrodes exposed through the one or more laser-cut windows.

In various embodiments, the plurality of elastomer layers comprises a second elastomer layer and a third elastomer layer, the second and third elastomer layers sandwiching the laser-cut polymer layer. In various embodiments, the elastomer layers are polyurethane layers (e.g., thermoplastic polyurethane (TPU) layers). In various embodiments, the flex-PCB comprises one or more serpentine conductive traces. In various embodiments, the device comprises a soft robotic sensor array (SRSA). In various embodiments, the device is an SRSA actuator. In various embodiments, the device is a hydraulic actuator. In various embodiments, the flex-PCB is a dual-layer flex-PCB.

### BRIEF DESCRIPTION OF THE DRAWINGS

FIG. 1A is a schematic image of a layered stack up for a soft robotic sensor array (SRSA) actuator, according to various illustrative embodiments of the disclosure: thermoplastic polyurethane (TPU), flexible printed circuit board (flex-PCB), polyvinyl alcohol (PVA), soft robotic sensing array (SRSA).

FIG. 1B are images of a laser postprocessed SRSA actuator before actuation (on left) and after actuation (on right), according to various illustrative embodiments of the disclosure.

FIG. 2 provides: at (A), a two-dimensional (2D) CAD design of dual-layer flex-PCB, according to various illustrative embodiments of the disclosure; at (B), an image of an unprocessed dual-layer flex-PCB coated with an electroless nickel immersion gold (ENIG) finish, according to various illustrative embodiments of the disclosure; and at (C), a processed dual-layer flex-PCB ENIG actuator, according to various illustrative embodiments of the disclosure.

FIG. 3 provides schematics of various zones for dual-layer flex-PCB, according to various illustrative embodiments of the disclosure. At (A) are provided schematics of four possible flex-PCB configurations: I) multiple serpentines on both the top and bottom layers, II) multiple serpentines on both top and bottom layers, plus a single layer serpentine on either layer, III) single serpentine on both the top and bottom layers, and VI) single serpentine on both either the top or bottom layer. At (B) is provided an image of a dual-layer flex-PCB. At (C) is provided an image of a laser cutting path with color-coded zones corresponding to laser power values: I) Red 11%, II) Blue 8%, III) Green 10%, and VI) Magenta 8%. Laser speed: 20%.

FIGS. 4A-4H correspond to optimized laser postprocessing parameters for all zones for dual-layer flex-PCBs, according to various illustrative embodiments of the disclosure. FIGS. 4A and 4B depict a first example configuration with multiple serpentines on both the top (4A) and bottom (4B) layers; FIGS. 4C and 4D depict a second example configuration with multiple serpentines on both top (4C) and bottom (4D) layers, plus a single layer serpentine on either layer; FIGS. 4E and 4F depict a third example configuration with a single serpentine on both the top (4E) and bottom (4F) layer; and FIGS. 4G and 4H depict a fourth example configuration with a single serpentine on either the top (4G) or bottom (4H) layer.

FIG. 5 provides input waveforms and measured waveforms using 16 sensors on the SRSA in saline water, according to various illustrative embodiments of the disclosure. The input waveform has a voltage of 20 millivolts (mV) and a pulse width of 50 milliseconds (ms).

FIGS. 6A-6D provide in vivo validation of electronic readings, according to various illustrative embodiments of the disclosure. FIG. 6A depicts a flex-PCB SRSA mounted on a 3D printed sample holder, FIG. 6B depicts an exposed leporine heart before collecting signals, FIG. 6C depicts an SRSA mounted on a 3D printed holder in contact with leporine heart, and FIG. 6D provides acquired signal using flex-PCB ENIG with a 500 Hz sampling rate.

FIG. 7 provides an image of a dual-layer flex-PCB before and after laser postprocessing at optimized power values, according to various illustrative embodiments of the disclosure. Laser speed: 20%.

FIG. 8A-8L provides microscope images for the different laser postprocessed flex-PCB regions under different laser power values, according to various illustrative embodiments of the disclosure.

### DETAILED DESCRIPTION

For the purposes of reading the description of the various implementations and techniques described herein, the following brief descriptions of the sections of the Specification may be helpful. Although cardiac mapping is discussed as a potential application, the disclosed approach is not so limited, and may extend to other applications as well, such as encephalographic applications or other studies of bioelectrical activity and mapping thereof.

In various embodiments, presented herein is an approach to creating stretchable electronics from dual-layer flex printed circuit boards (flex-PCBs) as a platform for soft robotic sensor arrays (SRSAs) for electrical activity mapping applications, such as cardiac voltage mapping applications. There is a crucial need for devices that utilize multiple sensors and provide high performance signal acquisition for cardiac mapping. Single-layer flex-PCB can be postprocessed to create a stretchable electronic sensing array. An example detailed fabrication process for creating a dual-layer multielectrode flex-PCB SRSA is presented, along with relevant parameters to achieve optimal postprocessing with a laser cutter according to various potential implementations. The ability of a dual-layer flex-PCB SRSA to acquire electrical signals is demonstrated both in vitro as well as in vivo on a Leporine cardiac surface. These SRSAs could be extended into full-chamber cardiac mapping catheter applications. The disclosed results demonstrate, in example embodiments, the scalable use of dual-layer flex-PCB for stretchable electronics.

### 1: Introduction to Example Embodiments

Cardiac mapping is a procedure used to characterize abnormal electrical activity in patients with cardiac arrhythmia, allowing operators to identify and treat the source of the arrhythmia using cardiac ablation. This can be performed using various techniques such as electroanatomic mapping, which uses a combination of electrical signals and imaging to create a detailed map of the heart's electrical activity. Once the location of the abnormal activity is identified, the patient can then be treated; a separate ablation catheter is inserted and energy is applied locally (e.g., radiofrequency, electroporation, cryothermal) to destroy the problematic tissue and restore a normal cardiac rhythm. Additionally, non-invasive methods such as electrocardiography, echocardiography, magnetic resonance imaging, computed tomography, and other imaging modalities can be used in conjunction with mapping to help localize the arrhythmia. Minimally invasive diagnostic catheters with one or many sensors at the tip allow electrophysiologists to collect electrical measurements from the heart's interior surface. By articulating the catheter, the sensors are moved around the cardiac surface. Signal and the location of the catheter at the time of collection can be aggregated to provide a more a comprehensive picture of the electrical activity. Mapping the electrical activity of the heart is a critical component for the diagnosis and treatment of arrhythmia.

The use of stretchable electrodes arrays allows for more conformable mapping and for simultaneous acquisition of electrical signals. This can lead to a more accurate and detailed map of cardiac arrhythmia. More broadly, a wide variety of demonstrations have shown the potential of utilizing structured 2D/3D electrodes to provide highly conformal sensing for a wide variety of biomedical applications.

Further, the use of high precision laser-based cutting and/or micromachining techniques have been used to create single and multilayer electronics with 3D and/or conformable systems. One team demonstrated personalized and digitally manufactured wireless biosensors for indefinite collection of high-fidelity biosignals. The researchers used laser engraving to pattern flexible circuit boards. The resulting devices were found to have high sensitivity and accuracy for measuring physiological signals. Another team created a fully implantable cardiac stimulation and recording devices. The researchers used a laser cutter to pattern flexible circuit boards and then assembled the devices into a 3D structure. The resulting device was found to have high accuracy for pacing and defibrillation in animal models. Yet another team developed graphene e-tattoos for unobtrusive ambulatory electrodermal activity sensing on the palm. The researchers used a laser cutter to pattern graphene and serpentine ribbons, resulting in a conformal and stretchable device that can measure electrodermal activity with high sensitivity and accuracy.

Another approach, based on thermal masking, allows for self-aligned removal of Kapton to eliminate Kapton from flex-PCB substrates, while preserving it underneath electrical traces. This allows for selective substrate removal without need for alignment and registration, for low cost, scalable creation of stretchable electrics. However, prior demonstrations were limited to single layer flex-PCBs. There is a need for higher densities of electrodes to achieve higher resolution mapping. Example embodiments may employ self-aligned laser based postprocessing techniques to dual and multi-layer flexible printed circuit boards (flex-PCBs). The use of dual-layer flex-PCBs allows for increased electrode densities as well as the ability to route signals past one another utilizing vias. Prior demonstrations illustrate a laser-based postprocessing technique that utilizes thermal masking. This approach, when utilized in conjunction with serpentine electrical traces, can yield stretchable electronic configurations that can accommodate up to ~30% strain. While this process has been successfully demonstrated in single layer flex-PCB designs, additional considerations arise when processing dual layer flex-PCBs.

Laser-based postprocessing of flex-PCBs is a versatile and precise method that can be used to convert inelastic flex-PCBs into stretchable electrode arrays. The laser postprocessing cutting technique using a thermal masking approach can be used to produce stretchable electronics. The technique involves using a laser to selectively remove insulation layers from a single-layer flex-PCB. This, in conjunction with the use of serpentine electrode designs, increases the stretchability of the electronics by allowing the metallic traces to expand and contract without being constrained by the insulation layers. However, because insulating layers are preserved under the metallic traces, when arrays are integrated with elastomeric substrates (e.g., silicone, polyurethane), the resulting structure is mechanically graded in its thickness, dramatically enhancing the stretchability of the resulting features. This approach provides for a scalable way to produce stretchable electronics. This method can provide a cost-effective and efficient way to produce stretchable electronics for various applications such as wearable devices, medical implants, and robotics.

Various embodiments of this disclosure provide an approach for creating stretchable electronics from dual-layer flex-PCB with serpentine sensor array designs. These stretchable electronics can be used, for example, as a platform for soft robotic sensor arrays (SRSA) for cardiac mapping. There is a crucial need for devices that can embed multiple sensors and provide better signal acquisition for cardiac mapping applications. Various embodiments provide a fabrication process of a dual-layer multielectrode flex-PCB soft robotic sensor array (SRSA). Various embodiments use self-aligned laser-postprocessed in multi-layer flex-PCB materials. Example embodiments of the disclosed arrays can be utilized for cardiac mapping, as discussed below by showing the SRSA's ability to acquire electrocardiograms both in vitro as well as in vivo on a leporine heart.

### 2: Illustrative Experiments

### 2.1: Materials and Instrumentation

### 2.1.1: Fabrication Stack Up for the Soft Robotic Sensor Array Actuator

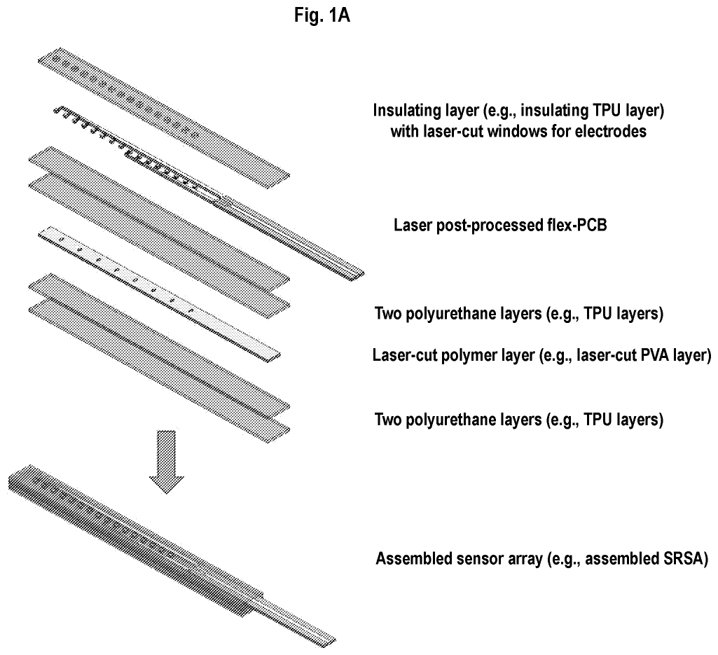

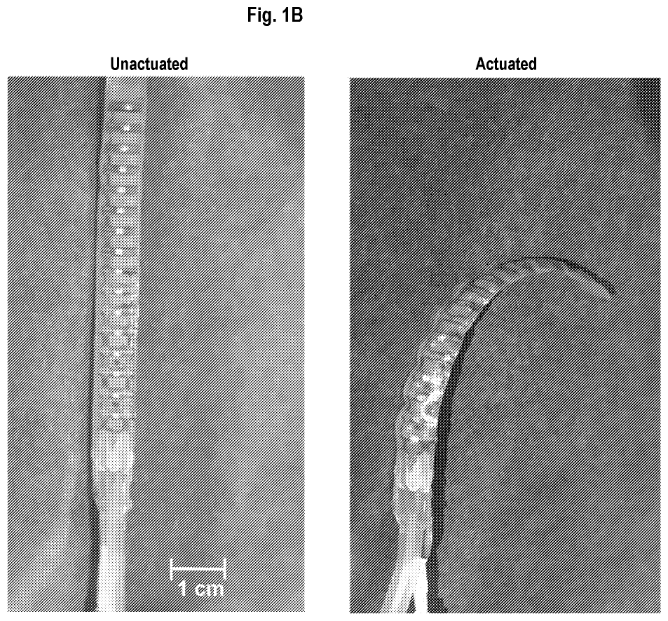

Example stack-ups of the dual-layer flex-PCB SRSA layers used in various experiments are presented in FIGS. 1A and 1B. The inelastic flex-PCB was designed and sourced from a conventional flex-PCB vendor (EPEC Engineered technologies, New Bedford, MA, USA). The fabrication stack up process allows for the integration of the flex-PCB electronics into an actuator. The components used are designed using Rhinoceros 3D software (Rhino 6, North Seattle, WA, USA). All the components used are prepared from sheets using a laser cutter (VL300 Universal laser systems, 30 W-600×300 mm) Universal laser systems, Scottsdale, Arizona, USA. This laser can cut and engrave a variety of materials up to 5 mm thick and engrave many materials including wood, plastics, metals, stone, and glass.

To fabricate the soft robotic sensor array (SRSA) actuator, several steps have been followed in demonstrative experiments. As shown in FIG. 1A, the PVA (polyvinyl alcohol) was laser cut into a suitable dimension to be sandwiched between the two layers of the green thermoplastic polyurethane. The purpose of using PVA is to avoid the bonding of the TPU layer and to serve as a sacrificial layer allowing the facilitation of the liquid flow during the hydraulic actuation. As depicted in FIG. 1A, the flex-PCBs were laser postprocessed before integrating the electronics with the soft robotic actuators. On top of the electronics, an additional layer of TPU is heat-pressed to encapsulate them. This layer contains windows to allow the electrodes to be exposed during the data acquisition. These different layers are assembled and heat pressed to induce bonding between polyurethane layers (FIG. 1A). Once an actuator is complete, it is infused with water to dissolve the PVA layers creating a sealed hydraulic actuator. FIG. 1B shows this actuator before and after actuation.

### 2.1.2: In Vivo Voltage Mapping

The in vivo voltage mapping was studied using an open-surgical approach to acquire electrogram signals in leporine models. The electrical signals were acquired using a National Instrument data acquisition system controlled by a LabView homemade software (NI-6225, National Instruments Corporation, Austin, TX, USA). The leporine was anesthetized first and the chest was opened to expose the heart. The pericardial sac was stripped to allow access to the epicardial surface. Once the heart is exposed, the device is moved into contact with the cardiac surface to collect electrical signals. A 3D printed handle holder made from VeroClear material (Stratasys, Eden Prairie, MN, USA) with two slots allowed the actuators to expose only one specific electrode to the leporine heart surface. The handle also allows for irrigation with saline water to keep the heart surface moist, which helps to reduce friction, minimize damage to the cardiac tissue, and ensure the surface of the heart does not dry out. This not only enhances the safety of the procedure but also allows for more accurate and reliable results. These electrograms are postprocessed during and after the procedure to evaluate their quality. The leporine models remain under anesthesia for the duration of the recording and at the end of the recording.

### 3: Results and Discussion

### 3.1: Laser Postprocessing of Dual-Layer Flex-PCB Sensors

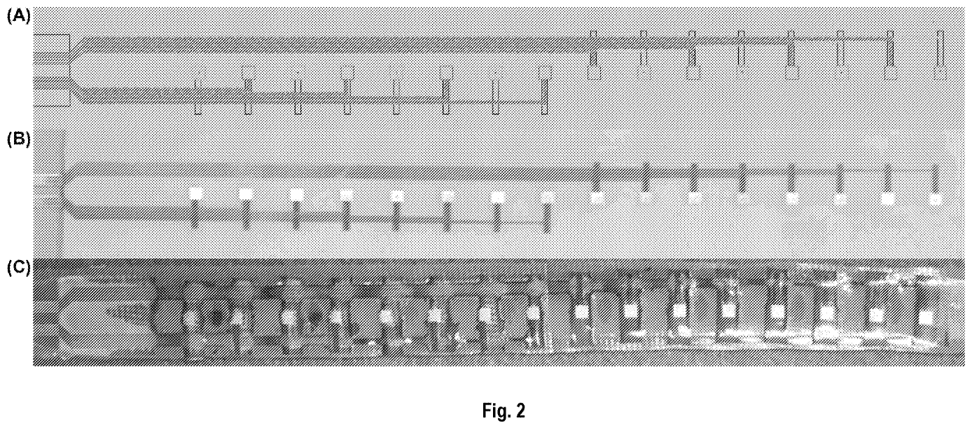

FIG. 2 presents the dual-layer flex-PCB sensor arrays CAD design. As shown at (A), a 2D CAD design presents the dual-layer flex-PCB containing copper serpentine traces for both layers, an in between polyimide layer for insulation and 16 electrodes. In the regions where the density of electrical connects is defined by the limits of the flex-PCB fabrication, the dual layer design allows for twice the density of traces relative to a single layer design. At (B), FIG. 2 shows the real image of the unprocessed flex-PCB. This type of actuator is made of a flex-PCB that has been coated with Electroless Nickel Immersion Gold (ENIG) finish, which provides a smooth and corrosion-resistant surface. At (C), FIG. 2 shows the prepared laser postprocessed dual-layer flex-PCB actuator that will be used for the in vitro and in vivo voltage mapping experiments described in the sections below. The flex-PCBs are covered with a layer of green thermoplastic polyurethane (TPU) to provide insulation and allow the actuation of the flex-PCB.

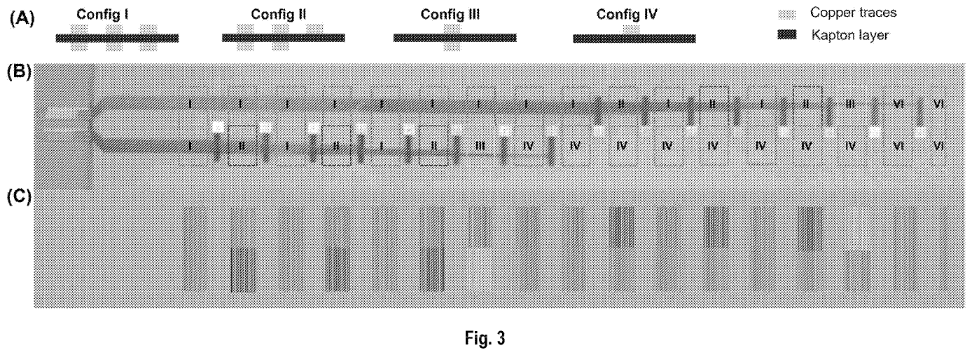

In various embodiments, the laser postprocessing of the flex-PCB stretchable electronics is essential for actuator functionality; unprocessed flex-PCB is too inextensible to accommodate the deformations associated with actuation. In this context, various embodiments employ a laser-based postprocessing that allows for scalable fabrication of stretchable sensor arrays (FIG. 3 at (A)). The dual-layer flex-PCBs were subjected to ultrasonication for 20 minutes and subsequently underwent a visual inspection to verify removal of Kapton from between traces. Additionally, specialized equipment such as a microscope or a camera with high magnification is used to inspect the board's surface. Any remaining Kapton is carefully removed using appropriate tools such as a pair of tweezers, taking care not to damage the underlying traces or components. It is worth noting that this successfully eliminated the Kapton between the copper traces in all areas except for configuration II, where the removal was only partial.

FIG. 3 shows a schematic of various zones for dual-layer flex-PCB. FIG. 3 marks the different laser-cutting paths with color-coded zones. The power required to optimally remove the polyimide, while preserving it below the traces, depends on the density of traces which conduct heat away from the laser spot and reduce the temperature of the heating zone. It is noted that the dual-layer flex-PCB used in this study has different regions that require different laser power values than those needed for processing single-layer flex-PCBs. To address this, various embodiments can divide the postprocessed regions into four different configurations based on their design and complexity. As can be seen in FIG. 3, these configurations include multiple serpentines on both top and bottom layers (ConFIG. I), a combination of multiple serpentines and single-layer serpentines on either layer (ConFIG. II), single serpentines on both top and bottom layers (ConFIG. III), and single serpentine on both either the top or bottom layer (ConFIG. IV). Table 1 provides a summary of the different scenarios for the laser postprocessing of the dual-layer flex-PCB ENIG. However, in the case of configuration II, where single-layer and dual-layer traces are situated close to each other, the dual-layer sections could not be optimally postprocessed without potentially damaging the single-sided traces. This is because dual-layer traces are more efficient in transmitting heat from the laser spot and require higher powers to ensure complete removal of Kapton material from between the traces.

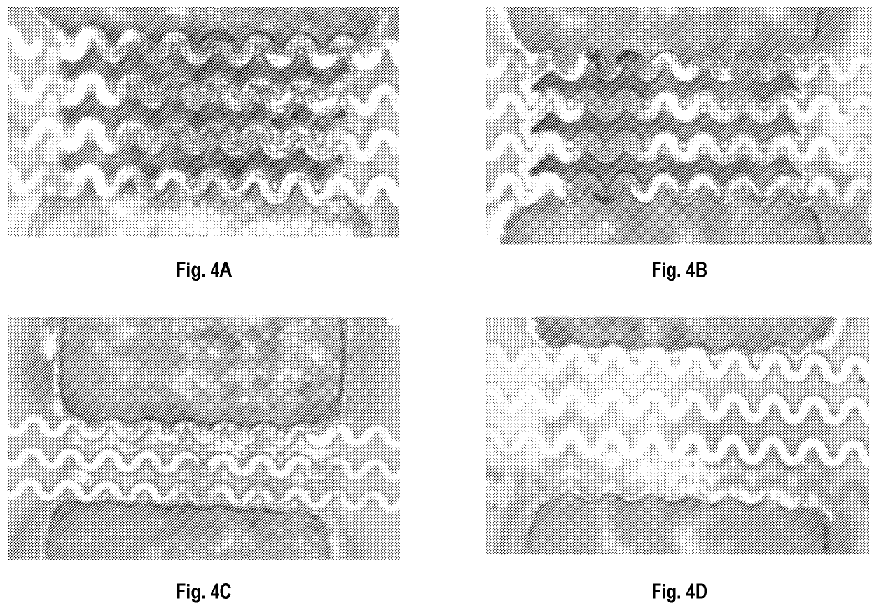

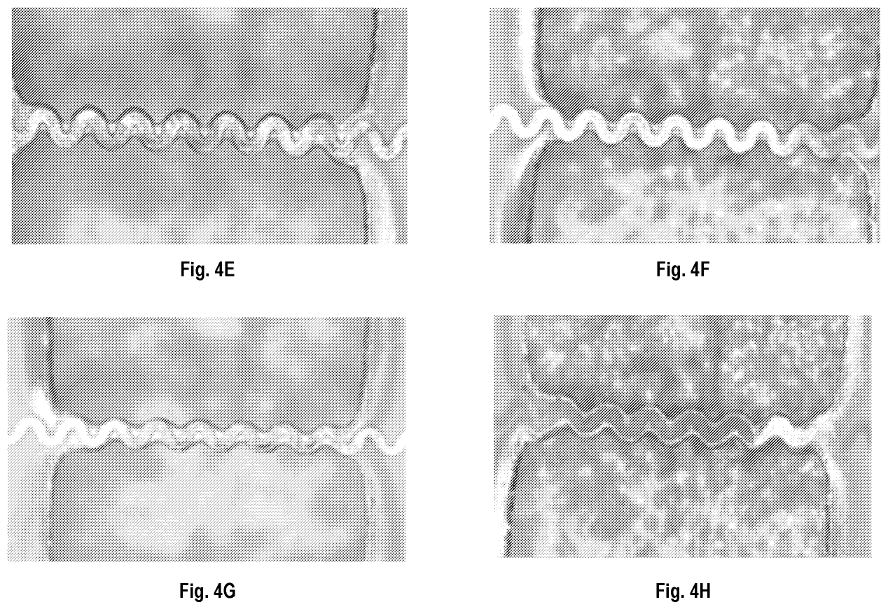

TABLE 1
Optimized laser postprocessing parameters
for all zones for dual-layer flex-PCB

| | Laser post- | Microscope | Microscope |
|---|---|---|---|
| Configuration | processing/optimized | Top | Bottom |
| Type | conditions | view image | view image |
| I | Multiple serpentines | FIG. 4A | FIG. 4B |
| | on both the top and | | |
| | bottom layers | | |
| | Red 11% | | |
| II | Multiple serpentines | FIG. 4C | FIG. 4D |
| | on both top and bottom | | |
| | layers, plus a single | | |
| | layer serpentine on | | |
| | either layer | | |
| | Blue 8% | | |
| III | Single serpentine on | FIG. 4E | FIG. 4F |
| | both the top and | | |
| | bottom layer | | |
| | Green 10% | | |
| IV | Single serpentine on | FIG. 4G | FIG. 4H |
| | both either the top | | |
| | or bottom layer | | |
| | Magenta 8% | | |

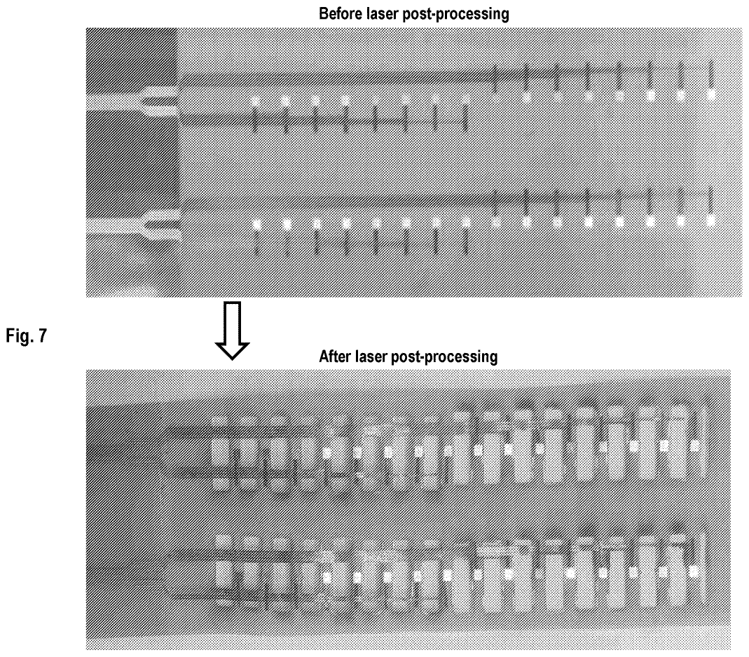

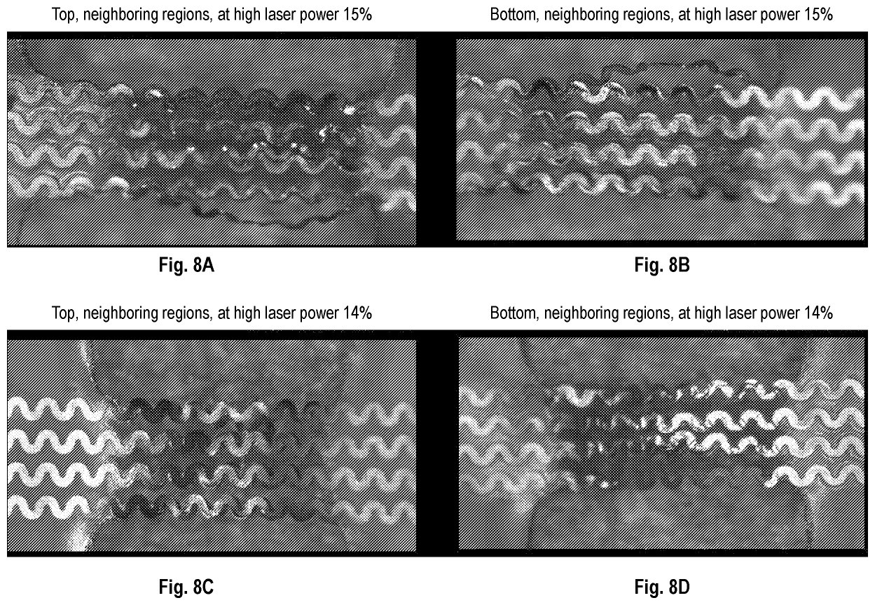

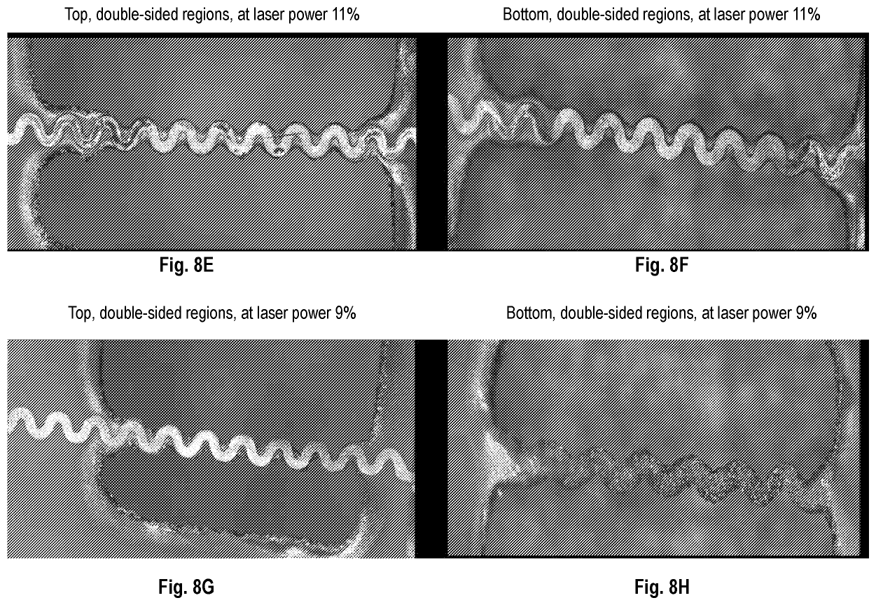

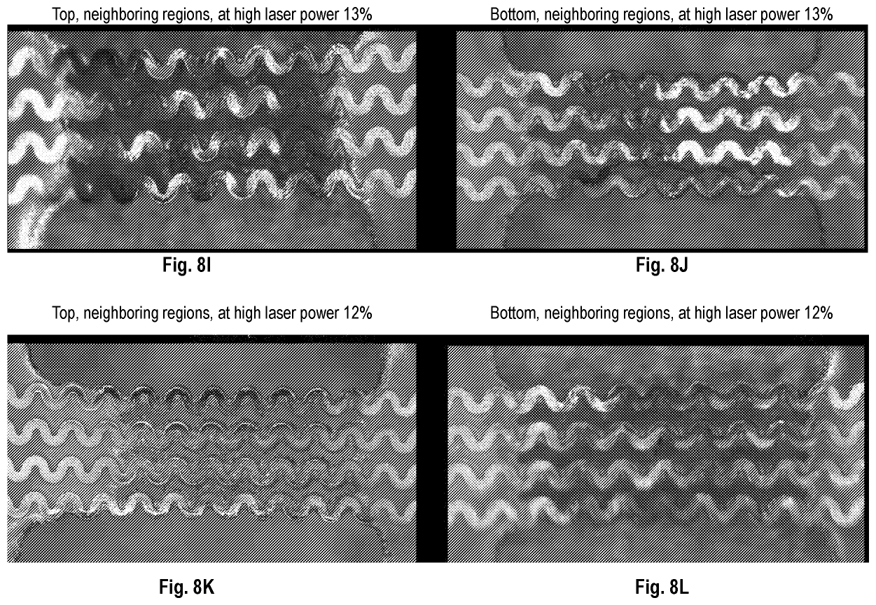

It is noted that, in various embodiments, designs that do not include single sided traces could be employed to mitigate this effect. Additionally, in example embodiments, single sided traces could be incorporated such that they require the same conditions to postprocess as dual layer traces, by adding a “dummy” bottom layer trace that follows the same path as the top layer trace but does not connect electrically to any electrodes. The values of laser postprocessing presented in this study are optimized (see FIGS. 7 and 8A-8L). Moreover, embodiments of this laser postprocessing allow for the preservation of the polyimide material underneath the copper traces resulting in a mechanically graded structure (in the z-axis), which can improve durability relative to similar traces without intermediate stiffness supports. As demonstrated, example embodiments can utilize this method with a multilayer flex-PCB, while achieving the same self-aligned material removal. This can provide the potential to utilize this embodiments of the disclosed approach to scalably convert circuits to stretchable arrays with more complex circuits designs that utilize overlapping traces and vias. The preservation of polyimide material and the use of laser post-processing method results in a high-quality flex-PCB that is durable, robust, and capable of meeting the demands of various electronic applications.

Disclosed herein are example embodiments of a methodology for laser postprocessing of the dual-layer flex-PCB ENIG. Identified herein are different configurations for the postprocessing of different regions of the dual-layer flex-PCB.

In various embodiments, an advantage of self-aligned laser-postprocessed dual-layer flex-PCBs is the ability to create stretchable electronics that can acquire signals from multiple sensors. This is particularly useful for applications in which multiple sensors are better suited to accurately mapping electrical activity, such as bioelectrical activity of an organ. Various embodiments can be employed for such applications as cardiac mapping, where multiple sensors are needed to accurately map the electrical activity of the heart. Additionally, in various embodiments, the use of dual-layer flex-PCBs allows for greater flexibility and stretchability. Moreover, in example embodiments, the precise alignment also allows for the creation of multielectrode arrays that can capture a larger area of electrical activity in the heart. Further, the use of multilayer designs that incorporate vias allow, in various embodiments, for more advanced circuits where traces can overlap on different layers. Thus, this demonstration provides broader complexity. The disclosed methodology can be used by researchers and engineers working on the development of flexible and stretchable electronics.

### 3.2: In Vitro Voltage Mapping Evaluations of SRSA

The as-prepared SRSA actuator's capability to perform voltage mapping has been assessed in saline water. Indeed, the in vitro voltage acquisition was carried out using a single array of sensors that contains 16 electrodes of the flex-PCB ENIG actuator.

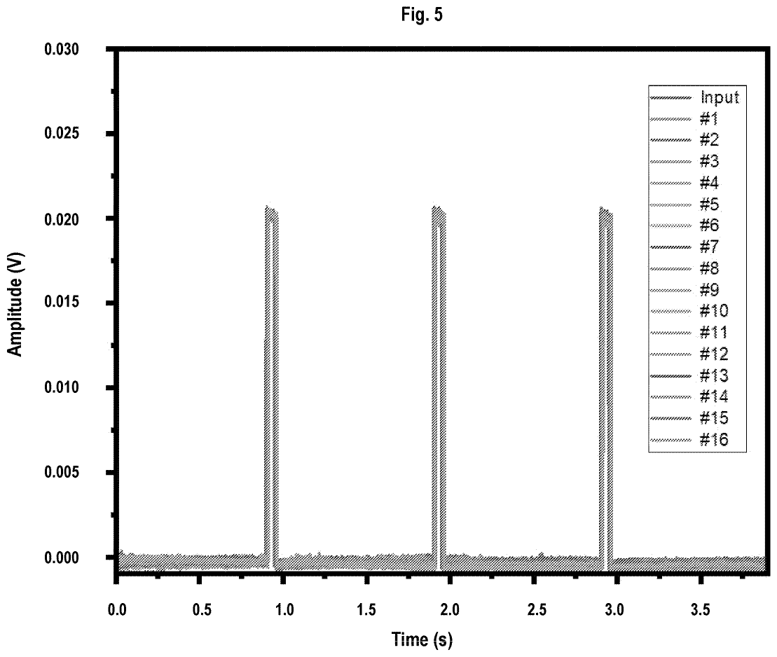

The in vitro study of the developed SRSA actuator was conducted to evaluate their performance in comparison to baseline measurements from an oscilloscope. The study involved taking simultaneous measurements using the oscilloscope at a frequency of 1 Hertz (Hz), an input amplitude of 20 millivolts (mV), and a pulse width of 50 milliseconds (ms). The choice of these experimental parameters is comparable to that of a realistic electrogram. The results of the study were represented in FIG. 5, which shows the sensors data for low voltage and low pulse width in standard saline solution. All 16 electrodes in the SRSA exhibit similar response for the input voltage as shown in FIG. 5. The data demonstrates the SRSA actuator's ability to effectively read electrograms, indicating their usability in further research.

### 3.3: Validation of Electronic Readings In Vivo Measurements

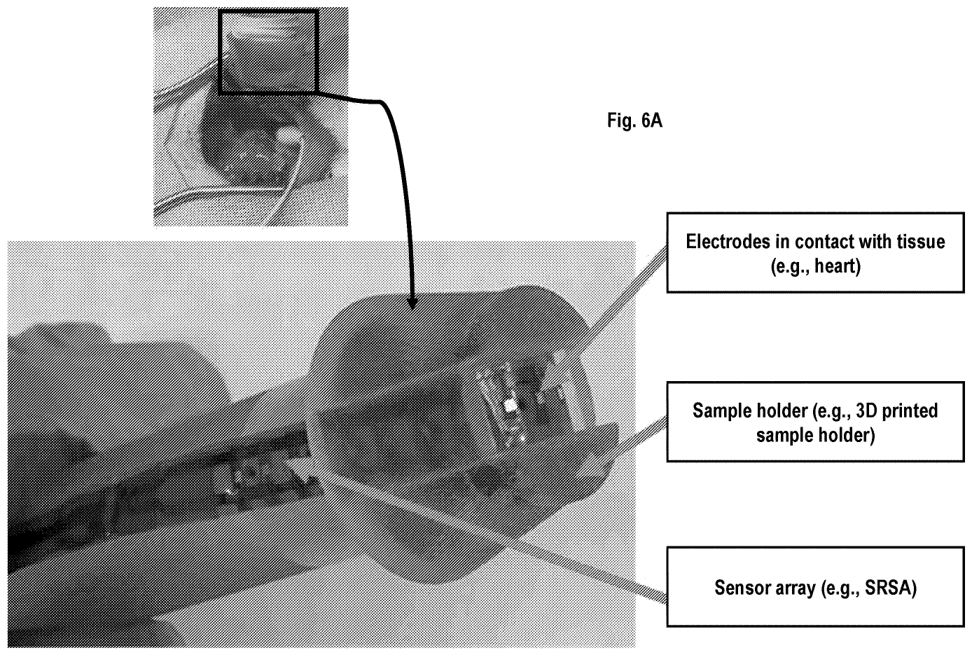

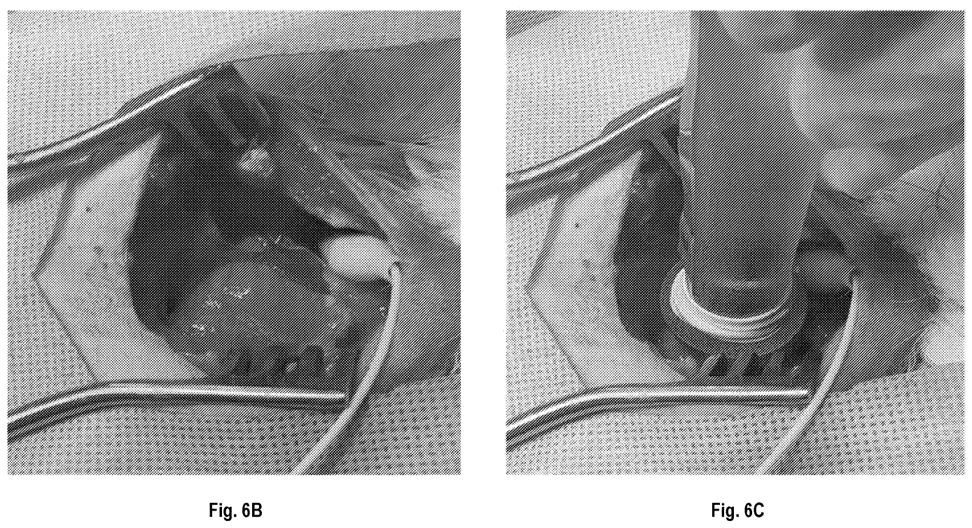

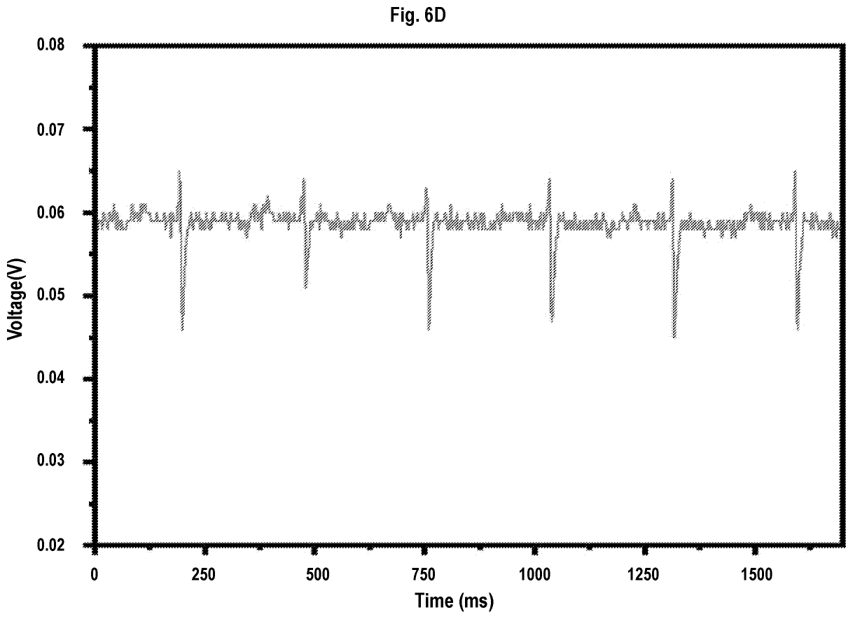

In demonstrative experiments, leporine animal models were used to perform the in vivo measurements using a smaller version of our device and/or only a subset of the sensors on the larger device. The single flex-PCB SRSA was mounted on a 3D printed sample holder electronic readings; the experiments used a single linear array of the dual flex-PCB ENIG actuator as shown in FIG. 6A to be used in contact with the leporine heart surface.

To achieve this goal, the experiments characterized signal acquisition from the epicardial surface of an exposed leporine heart. As described in the experimental section, an open surgical approach has been used to gain access to the epicardial surface to acquire electrogram signals (FIG. 6B). Once the heart is visualized, the SRSA mounted on the 3D printed handle holder is properly positioned to collect the electrical signals from the surface of the heart (FIG. 6C). The data acquisition system started the recording of the electrogram signals using the flex-PCB ENIG device under a 500 Hz sampling rate (FIG. 6D). The outcome of these experiments successfully confirmed the proper acquisition of electrogram signals from the leporine heart with high accuracy.

### 3.4: Discussion

Soft robotic features with embedded sensing exhibit high levels of conformability to complex tissue and anatomy. Various embodiments provide a new method for creating stretchable electronics using dual-layer flex-PCBs allowing for scalable fabrication of flexible circuits with higher electrode densities. Greater electrode density allows for more detailed mapping of electrical signals in, for example, the heart. Detailed are example embodiments of a process to produce a dual-layer multielectrode flex-PCB using laser based postprocessing and integration thereof into soft robotic sensor array (SRSA). The fabrication process for this SRSA is described in detail, including the design of the electrode pattern, the selection of materials, and the laser postprocessing of the flex-PCBs. Also provided are relevant parameters necessary to achieve optimal postprocessing of the flex-PCBs with a laser cutter. The ability of the dual-layer flex-PCB SRSA to acquire electrical signals both in vitro and in vivo was demonstrated on a leporine cardiac surface. This result indicates the usability of these SRSAs for full-chamber cardiac mapping catheter applications.

The disclosed approach represents an advancement towards the scalable use of dual-layer flex-PCBs for stretchable electronics. It provides the ability to scalably fabricate stretchable sensor designs with higher density. The use of dual-layer flex-PCBs as a platform for stretchable electronics provides a versatile and scalable solution for creating electronics that can conform to complex surfaces and detect electrical signals from multiple sensors simultaneously. Various embodiments can also be used to construct multilayer circuits with vias and interconnects to allow stretchable designs from a scalable components. The compatibility of the materials with biological tissues may be achieved by constructing dual-layer flex-PCB SRSA from medical grade polyurethanes and PCB materials. The proposed approach thus promotes development of stretchable electronics for biomedical applications, enabling development of new medical devices that can improve patient outcomes by providing accurate and high-performance signal acquisition for cardiac mapping and other biomedical applications.

Various embodiments of the disclosure demonstrate an approach for the fabrication of stretchable electronics using dual-layer flex-PCBs and laser postprocessing technique. The resulting stretchable SRSA actuators exhibited excellent performance in acquiring electrical signals in vitro and were further validated using a leporine model to assess the acquisition of electrograms from a real cardiac surface. Experiments confirmed that proper voltage mapping signals could be acquired with high accuracy, demonstrating the potential of these stretchable electronics for use in various medical applications. These approaches may be applicable to larger multiactuator cardiac mapping system. This represents a meaningful demonstration of a highly conformable device design, showing the benefits that enhanced conformability provide. The disclosed use of dual-layer flex-PCBs and laser postprocessing thus provide an approach for the fabrication of stretchable electronics with a wide range of applications in medical diagnostics and treatment.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims. In some example embodiments, these terms allow for a plus-or-minus deviation of 15 percent, 10 percent, or 5 percent.

It should be noted that the terms “exemplary,” “example,” “potential,” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It is important to note that the construction and arrangement of the devices, assemblies, and steps as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principles of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the terms “approximately,” “about,” “substantially,” and similar terms in reference to a number or value is generally taken to include numbers or values that fall within a range of 1%, 5%, or 10% in either direction (greater than or less than) of the number or value unless otherwise stated or otherwise evident from the context (except where such number would be less than 0% or exceed 100% of a possible value).

As used herein, the terms “individual”, “patient”, or “subject” are used interchangeably and refer to an individual organism, a vertebrate, a mammal, or a human. In a preferred embodiment, the individual, patient or subject is a human.

While this specification contains many specific implementation details, these should not be construed as limitations on the scope of any implementations or of what may be claimed, but rather as descriptions of features specific to particular implementations of the systems and methods described herein. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed only in connection with one implementation are not intended to be excluded from a similar role in other implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. Although the examples provided herein relate to controlling the display of content of information resources, the systems and methods described herein can include applied to other environments. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

Having described certain embodiments of methods and systems, it will now become apparent to one of skill in the art that other embodiments incorporating the concepts of the disclosure may be used. Therefore, the disclosure should not be limited to certain embodiments, but rather should be limited only by the spirit and scope of the below examples and claims.

### EXAMPLES

Some non-limiting examples are disclosed below, in order to represent illustrative embodiments, which one skilled in the art will understand may be further modified, combined, constrained, etc. according to the entirety of this disclosure.

Embodiment A1: A method of manufacturing a device, the method comprising: sandwiching a sacrificial layer between a first non-sacrificial layer and a second non-sacrificial layer; encapsulating, in a third non-sacrificial layer, a laser postprocessed flexible printed circuit board (flex-PCB) comprising one or more electrodes, wherein the third non-sacrificial layer comprises one or more windows to expose the one or more electrodes; bonding the first non-sacrificial layer, the second non-sacrificial layer, and the third non-sacrificial layer together; and sacrificing the sacrificial layer.

Embodiment A2: The method of Embodiment A1, wherein the device is a soft robotic sensor array (SRSA) actuator.

Embodiment A3: The method of either Embodiment A1 or A2, further comprising postprocessing the flex-PCB.

Embodiment A4: The method of any of Embodiments A1-A3, wherein postprocessing the flex-PCB comprises subjecting the flex-PCB to ultrasonication.

Embodiment A5: The method of any of Embodiments A1-A4, wherein the flex-PCB comprises a conductive trace between two insulating layers.

Embodiment A6: The method of any of Embodiments A1-A5, wherein the method further comprises postprocessing the flex-PCB.

Embodiment A7: The method of any of Embodiments A1-A6, wherein postprocessing the flex-PCB comprises applying a laser to remove a first portion of the insulating layer without removing a second portion of the insulating layer.

Embodiment A8: The method of any of Embodiments A1-A6, wherein postprocessing the flex-PCB comprises applying a first laser power level at a first region of the flex-PCB and applying a second laser power level at a second region of the flex-PCB.

Embodiment A9: The method of any of Embodiments A1-A8, wherein the sacrificial layer is a water-soluble layer.

Embodiment A10: The method of any of Embodiments A1-A9, wherein the method further comprises infusing the bonded first, second, and third non-sacrificial layers in water to dissolve the sacrificial layer.

Embodiment A11: The method of any of Embodiments A1-A10, wherein encapsulating the flex-PCB in the third non-sacrificial layer comprises permanent adhesion by chemical means, heat-pressing, and/or welding the third non-sacrificial layer to the flex-PCB.

Embodiment A12: The method of any of Embodiments A1-A11, further comprising laser-cutting the one or more windows in the third non-sacrificial layer.

Embodiment A13: The method of any of Embodiments A1-A12, wherein bonding the first, second, and third non-sacrificial layers comprises permanent adhesion by chemical means, heat-pressing, and/or welding the first, second, and third non-sacrificial layers together.

Embodiment A14: The method of any of Embodiments A1-A13, further comprising using the device.

Embodiment A15: The method of any of Embodiments A1-A14, further comprising using the device to map electrical activity.

Embodiment A16: The method of any of Embodiments A1-A15, further comprising mapping bioelectrical activity of an organ.

Embodiment A17: The method of any of Embodiments A1-A16, further comprising mapping bioelectrical activity of a heart.

Embodiment A18: The method of any of Embodiments A1-A17, further comprising mapping bioelectrical activity of a brain.

Embodiment A19: A device produced through any of the methods of any of Embodiments A1-A13.

Embodiment A20: A method of using the device of Embodiment A19.

Embodiment B1: A method of postprocessing a multilayer flexible PCB (flex-PCB) to increase deformability of the flex-PCB, wherein the flex-PCB comprises a conductive trace at least partially embedded in an insulating layer.

Embodiment B2: The method of Embodiment B1, comprising applying a laser to the flex-PCB so as to remove a first portion of the insulating layer without removing a second portion of the insulating layer.

Embodiment B3: The method of either Embodiment B1 or B2, wherein the second portion of the insulating layer is below the conductive trace.

Embodiment B4: The method of any of Embodiments B1-B3, wherein applying the laser to the flex-PCB comprises applying a first laser power at a first region of the flex-PCB and a second laser power at a second region of the flex-PCB.

Embodiment B5: The method of any of Embodiments B1-B4, further comprising subjecting the flex-PCB to ultrasonication prior to applying the laser to the flex-PCB.

Embodiment B6: The method of any of Embodiments B1-B5, further comprising manufacturing a device comprising the flex-PCB of any of Embodiments B1-B5.

Embodiment B7: The method of any of Embodiments B1-B6, further comprising using the flex-PCB in a device.

Embodiment B8: A flex-PCB produced through any of the methods of any of Embodiments B1-B7.

Embodiment B9: A device comprising any flex-PCB of Embodiment B8.

Embodiment B10: A device produced through any of the methods of any of Embodiments B1-B7.

Embodiment C1: A device comprising: a plurality of elastomer layers, the plurality of elastomer layers comprising a first elastomer layer with one or more laser-cut windows; and a laser postprocessed flexible printed circuit board (flex-PCB) encapsulated in the plurality of elastomer layers, wherein the flex-PCB includes one or more electrodes exposed through the one or more laser-cut windows.

Embodiment C2: The device of Embodiment C1, wherein the plurality of elastomer layers further comprises a second elastomer layer and a third elastomer layer, the second and third elastomer layers sandwiching the laser-cut polymer layer.

Embodiment C3: The device of either Embodiment C1 or C2, wherein the elastomer layers are thermoplastic polyurethane (TPU) layers.

Embodiment C4: The device of any of Embodiments C1-C3, wherein the flex-PCB comprises one or more serpentine conductive traces.

Embodiment C5: The device of any of Embodiments C1-C4, wherein the device comprises a sensor array.

Embodiment C6: The device of any of Embodiments C1-C5, wherein the device comprises a soft robotic sensor array (SRSA).

Embodiment C7: The device of any of Embodiments C1-C6, wherein the device is an SRSA actuator.

Embodiment C8: The device of any of Embodiments C1-C7, wherein the device is a hydraulic actuator.

Embodiment C9: The device of any of Embodiments C1-C8, wherein the flex-PCB is a multi-layer flex-PCB.

Embodiment C10: The device of any of Embodiments C1-C9, wherein the flex-PCB is a dual-layer flex-PCB.

Embodiment D1: A system and/or a device incorporating any of the above devices.

Embodiment E1: A system and/or a device for performing any of the above methods.

Embodiment F1: A method for fabricating any of the above devices or systems.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

What is claimed is:

1. A method of manufacturing a device, the method comprising:
sandwiching a sacrificial layer between a first non-sacrificial layer and a second non-sacrificial layer;
encapsulating, in a third non-sacrificial layer, a laser postprocessed flexible printed circuit board (flex-PCB) comprising one or more electrodes, wherein the third non-sacrificial layer comprises one or more windows to expose the one or more electrodes;
bonding the first non-sacrificial layer, the second non-sacrificial layer, and the third non-sacrificial layer together; and
sacrificing the sacrificial layer.

2. The method of claim 1, wherein the device is a soft robotic sensor array (SRSA) actuator.

3. The method of claim 1, further comprising postprocessing the flex-PCB, wherein postprocessing the flex-PCB comprises subjecting the flex-PCB to ultrasonication.

4. The method of claim 1, wherein the flex-PCB comprises a conductive trace between two insulating layers, wherein the method further comprises postprocessing the flex-PCB, and wherein postprocessing the flex-PCB comprises applying a laser to remove a first portion of the insulating layer without removing a second portion of the insulating layer.

5. The method of claim 1, further comprising postprocessing the flex-PCB, wherein postprocessing the flex-PCB comprises applying a first laser power level at a first region of the flex-PCB and applying a second laser power level at a second region of the flex-PCB.

6. The method of claim 1, wherein the sacrificial layer is a water-soluble layer, and wherein the method further comprises infusing the bonded first, second, and third non-sacrificial layers in water to dissolve the sacrificial layer.

7. The method of claim 1, wherein encapsulating the flex-PCB in the third non-sacrificial layer comprises permanently adhesion by chemical means, heat-pressing, and/or welding the third non-sacrificial layer to the flex-PCB.

8. The method of claim 1, further comprising laser-cutting the one or more windows in the third non-sacrificial layer.

9. The method of claim 1, wherein bonding the first, second, and third non-sacrificial layers comprises permanently adhesion by chemical means, heat-pressing, and/or welding the first, second, and third non-sacrificial layers together.

10. A method of postprocessing a multilayer flexible PCB (flex-PCB) to increase deformability of the flex-PCB, the flex-PCB comprising a conductive trace at least partially embedded in an insulating layer, the method comprising:
applying a laser to the flex-PCB so as to remove a first portion of the insulating layer without removing a second portion of the insulating layer, wherein the second portion of the insulating layer is below the conductive trace.

11. The method of claim 10, wherein applying the laser to the flex-PCB comprises applying a first laser power at a first region of the flex-PCB and a second laser power at a second region of the flex-PCB.

12. The method of claim 10, further comprising subjecting the flex-PCB to ultrasonication prior to applying the laser to the flex-PCB.

13. A device comprising:
a plurality of elastomer layers, the plurality of elastomer layers comprising a first elastomer layer with one or more laser-cut windows; and
a laser postprocessed flexible printed circuit board (flex-PCB) encapsulated in the plurality of elastomer layers, wherein the flex-PCB includes one or more electrodes exposed through the one or more laser-cut windows.

14. The device of claim 13, wherein the plurality of elastomer layers further comprises a second elastomer layer and a third elastomer layer, the second and third elastomer layers sandwiching the laser-cut polymer layer.

15. The device of claim 13, wherein the elastomer layers are thermoplastic polyurethane (TPU) layers.

16. The device of claim 13, wherein the flex-PCB comprises one or more serpentine conductive traces.

17. The device of claim 13, wherein the device comprises a soft robotic sensor array (SRSA).

18. The device of claim 13, wherein the device is an SRSA actuator.

19. The device of claim 13, wherein the device is a hydraulic actuator.

20. The device of claim 13, wherein the flex-PCB is a multi-layer flex-PCB.
